(19) United States
(12) Patent Application Publication
(10) Pub. No.: US 2022/0000947 A1
(43) Pub. Date: Jan. 6, 2022

(54) PROBIOTIC COMBINATION FOR TREATMENT OF ALLERGIC DISORDERS

(71) Applicant: Société des Produits Nestlé S.A., Vevey (CH)

(72) Inventors: Jalil BENYACOUB, Epalinges (CH); Stéphane DUBOUX, St-Prex (CH); Gail CZARNECKI-MAULDEN, Edwardsville, IL (US); Guénolée Eliane Marie PRIOULT, Bern (CH); Gabriela BERGONZELLI DEGONDA, BUSSIGNY (CH); Elizabeth FORBES-BLOM, Epalinges (CH); Mario Noti, Vulliens (CH)

(21) Appl. No.: 17/292,993

(22) PCT Filed: Dec. 18, 2019
(86) PCT No.: PCT/EP19/86089

§ 371 Date: May 11, 2021

(30) Foreign Application Priority Data
Dec. 21, 2018 (EP) 18215094.6

(51) Int. Cl. A61K 35/745; A23L 33/135; A23K 10/16; A61P 37/08

(52) U.S. Cl. CPC A61K 35/745; A61P 37/08; A23K 10/16; A23L 33/135

(57) ABSTRACT

Disclosed is a probiotic combination comprising B longum CNCM I-2618 and B lactis CNCM I-3446 and —optionally further comprising B. longum ATCC BAA-999 for the treatment or prevention of an allergic disorder, or for the treatment or prevention of a food intolerance.

The present invention relates to a combination for the treatment or prophylaxis of allergic disorders including food allergies and/or food intolerances, and to compositions and methods employing the combination.

Allergies are among the most common health problems affecting the life of patients of all ages. Allergic diseases are nowadays recognized as an epidemic by the World Health Organization (WHO). The prevalence of allergies increased rapidly over the past decades. It has been estimated that over a third of the worldwide population is affected, to the extent that allergy has been considered as the new epidemic of the industrialized countries. The reasons for the steady increase in allergic diseases are not yet fully understood. Genetic background of the host is a prominent factor, and recently discovered genes have been shown to be associated with respiratory allergies/asthma and skin symptoms. Environmental factors such as lifestyle, pollution, decreasing family size, and reduction of microbial stimulation of the immune system in early life stage as a consequence of an improved hygienic situation seem to play an important role in the high prevalence and higher severity of allergic manifestations.

Allergic sensitization in childhood, especially in early childhood and especially to food allergens, is critical and of the highest interest as the development of an “allergic phenotype” or “atopy” has been shown to facilitate subsequent sensitization to other allergens. Hence allergies in childhood can be the first step of an allergic cascade leading to multiple allergies later in life, a process commonly referred to as the “Atopic March”. For example, children with persistent food hypersensitivity early in life have a dramatically increased risk to develop allergic rhinitis (hay fever) or asthma later in childhood. Children with milder forms of food hypersensitivity also have increased risk for development of respiratory allergies but to a lesser degree than children with persistent food hypersensitivity. Therefore, attenuating the severity of food hypersensitivity may be crucial for slowing down the “Atopic March”. In this context the management of allergic episodes and the prevention of allergies are, in childhood and infancy, of the highest importance.

The immune system of infants is actively developing all along the few first years of life. Acting on, preventing, avoiding, managing, reducing or modulating the allergic reactions in such young patients can influence their allergic profile not only in the short term but also longer term for later in life.

### Prevention of Allergies can be Achieved on Different Levels:

“Primary prevention” is the effect of preventing or reducing the risk of sensitization of patients to allergens, characterized by absence or reduced levels of allergen-specific IgE antibodies. Preventing or reducing sensitization will result in absence or reduction of allergic symptoms upon exposure to the same allergen. By modulating the way a patient gets sensitized in regard to one allergen or one group of allergens (primary prevention), the subsequent allergic response may also be modulated.

“Secondary prevention” is the effect of modulating the symptoms of allergies, i.e. the occurrence or intensity of the allergic reaction in patient already sensitized to one or several allergens when the patient is re-exposed to said allergen(s). By modulating the occurrence or intensity of the allergic symptoms (secondary prevention), the inconvenience associated with allergies is minimized.

Given these distinct concepts of allergy prevention it may be hypothesized that by virtue of their inherent mechanisms of action, some compounds might act solely at one or at both of these specific levels of prevention. Some may, for example, solely reduce the sensitization to a specific allergen (primary prevention), while other compounds may solely have an effect on the secondary prevention and reduce the severity of allergic reactions. Other compounds may be able to influence both sensitization and symptoms and thus are effective in promoting primary and secondary prevention.

Food allergens are among the first allergens that infants encounter in their early life: typically, cow's milk proteins may be encountered by infants not receiving exclusive breast-feeding. Milk-proteins are indeed among the most frequently observed causes for food allergy in infancy, followed by eggs and wheat proteins. In general, food allergies can manifest in cutaneous (rash, eczema, others) and gastrointestinal symptoms (abdominal cramps; pain, especially in the abdomen; vomiting) in infants and young children. Food allergies are the most common trigger of severe allergic reactions, which may lead to life-threatening anaphylaxis. Further sensitization and episodes of allergies can also appear when the infant/young child is exposed to a novel food such as cereals, vegetables, fruits, nuts or fish, and also to air-borne allergens such as pollen, house dust mites and animal dander. Adults are affected to a large extent by contact and respiratory allergies. Recent data from the WHO [Clark, M. J. and. Million, R. P (2009) Allergic rhinitis: market evolution, Nature Reviews, Drug Discovery, 8, p. 271-272] indicates that up to 30-40% of the world's population suffer from some form of respiratory allergy.

Animals, particularly small animals such as pets—and especially companion animals such as dogs and cats, may also suffer from food allergies and food intolerances, as well as environmental allergens. These typically manifest in similar symptoms to humans, e.g. gastrointestinal disturbances such as diarrhoea, vomiting and abdominal discomfort, and also dermatitis or pruritis. In small animals, particularly dogs, the most frequent cause of chronic diarrhoea is food-responsive enteropathy (diet-responsive enteropathy or food-responsive diarrhoea).

Immunotherapy has been employed for the treatment of asthma, allergic rhinitis and other allergic responses. Immunotherapy can reduce clinical symptoms and induce tolerance to allergens by immune response modulation. However, immunotherapy such as desensitization therapy, is not consistently effective for the treatment of food allergies, and even less so for respiratory allergies. Moreover, immunotherapy may induce serious systemic reactions when used in the treatment of food allergy such as nut allergy.

In animals, exclusion diets, novel protein diets (e.g. meat from atypical animal sources), hydrolyzed diets, and dietary challenge, have been used to treat food allergies, but treatments involving special diets may not be practical, especially for long term use and do not treat the underlying problem. Also, controlling the diet of an outdoor cat by elimination or other special diets is difficult, if not impossible. Management of a dietary challenge in an animal can take considerable time, effort and veterinary supervision, and may not be a practical solution.

Therefore, there still remains a need to develop new strategies for treating or preventing allergies, allergic reactions and/or symptoms in infants, children and adults, as well as in animals, in particular companion animals (pets), which may also suffer from food, airborne or contact allergies.

### Gastrointestinal Microbiota, Intestinal Barrier Integrity and the Immune System

Commensal gastrointestinal microbes constitute the earliest and most substantial stimulus for the development of the gut associated lymphoid tissue and associated immune system.

There is solid evidence from epidemiological studies that Western-type living conditions, e.g. reduced consumption of fermented food, substantial use of antibiotics and other drugs, and increased hygiene, are associated with the rise in allergic diseases. This links to the so-called “hygiene hypothesis” that suggests that a lack of exposure to microbial stimulus early in childhood is a major factor effecting the prevelance of allergic diseases. Indeed, epidemiological studies have demonstrated an association between the development of allergic diseases and disturbance of the gastrointestinal microbiota. Epidemological data show that atopic children have different intestinal flora compared with non-atopic children.

Such changes in the intestinal flora may also have a negative impact on the integrity of the intestinal barrier. Impaired barrier function, termed “leaky gut” has long been considered a predisposing factor for gastrointestinal diseases (Heyman, M. Eur. J. Gastroenterol. Hepatol. 17:1279-1285; Odenwald M, Nature Reviews Gastroenterology & Hepatology, (2017), (14), 9-21). As such, alterations in gut barrier integrity/function have multiple consequences facilitating the onset of numerous diseases depending on other hits and on genetic and epigenetic constellations. Food allergy patients often demonstrate with increased intestinal permeability, which correlates with the severity of their clinical symptoms (Ventura, M. T et al. 2006. Dig. Dis. Sci. 38:732-736). Preclinical animal models further provide corroborative evidence supporting a role for intestinal barrier dysfunction and leaky gut, predisposing to oral sensitization and subsequent development of food allergy. In addition, Western diet-induced alterations in intestinal permeability promote food allergen sensitization and clinical allergy symptoms in mice in response to dietary antigens (Hussain M. et al. J. Allergy Clin. Immunol. (2019). Probiotics represent one nutritional attempt to improve/reinforce intestinal barrier integrity and/or function (Ewaschuk J B et al., Am J Gastrointest Liver Physiol 2008 November; 295(5):G1025-34). Reinforcing intestinal barrier integrity by means of probiotic supplementation may thus prevent sensitization to oral allergens in at risk individuals. (Tulyeu J, Microorganisms. 2019 Oct. 16; 7(10).

Besides an established role of gut barrier function in allergen sensitization, uncontrolled immune responses towards dietary or environmental antigens foster the development of type-2 immune mediated allergic disorders. Probiotic cultures or mixes of probiotics have well known immunomodulatory properties that can prevent or alleviate allergic responses: for example, WO2006697949, describes a mix of probiotics that can decrease the risk of allergies due to wheat flour albumin and globulins (celiac disease).

### Role of Anti-Inflammatory Cytokines in Allergic Disorders and Intolerances

T regulatory (Treg) cells are critical for tolerance induction. Many chronic inflammatory diseases such as psoriasis, allergies and inflammatory bowel disease are considered to develop via a breakdown in tolerance. Failure to induce Treg activity has been demonstrated to lead to aberrant Th2 responses and the development of allergic disease. A number of anti-inflammatory cytokines such as IL-10 have been implicated in the prevention and resolution of allergic responses. Treg cells mediate their suppressive activity inter alia through secretion of anti-inflammatory cytokines, such as IL-10. IL-10 is a potent inhibitor of monocyte/macrophage function, suppressing pro-inflammatory cytokine production by antigen-presenting cells and T cells. IL-10 has potent anti-inflammatory effects. Underproduction of IL-10 from alveolar macrophages of atopic asthmatics has been reported. Moreover, IL-10 deficiency has been found in psoriasis, allergic contact dermatitis, inflammatory bowel disease and other inflammatory diseases. IL-10 has also been shown to inhibit allergen-induced airway inflammation in animal models of asthma, and underproduction of IL-10 in atopic asthmatics has been reported. Hadis, U., et al (Immunity (2011), 34(2), 237-246 showed that mice deficient in the chemokine (C-X30C motif) receptor 1 (CX3CR1), showed a substantially reduced production of IL-10 in intestinal macrophages, with the mice failing to show oral tolerance in an allergic diarrhea model.

Tiemessen, M. M, et al., J. Allergy Clin. Immunol. (2004), 113(5), 932-939 found production of IL-10 and activation of cow's milk-specific T-cell clones in non-allergic subjects, compared with cow's milk intolerant subjects, suggesting a key role for IL-10 in cow's milk allergy/intolerance.

Macrophages are tissue-based phagocytic cells derived from monocytes, which play an important role in the innate immune response. They are activated by microbial components and once activated, can themselves secrete both pro- and anti-inflammatory cytokines. He, F., et al (“Stimulation of the Secretion of Pro-Inflammatory Cytokines by Bifidobacterium Strains”-Microbiol. Immunol. (2002), 46(11), 781-785) investigated the ability of different bifidobacteria strains to affect the production of macrophage derived cytokines. They discovered that “adult type” bifidobacteria such as Bifidobacterium adolescentis and Bifidobacterium longum induced significantly more pro-inflammatory cytokine secretion than did “infant type” bifidobacteria such as Bifidobacterium bifidum, Bifidobacterium breve and Bifidobacterium infantis. In addition it was noted that B. adolescentis in particular did not stimulate production of the anti-inflammatory cytokine IL-10. They concluded that adult-type bifidobacteria may be more potent to amplify, but less able to down-regulate, the inflammatory response. However, more recently, attempts to identify the most promising anti-inflammatory probiotic strains for therapeutic use have indicated that taxonomic classification of a probiotic is not generally a reliable predictor of, e.g. the anti-inflammatory properties, of a particular probiotic strain.

### SUMMARY OF THE INVENTION

The present inventors have surprisingly discovered that a combination of two probiotic strains comprising a Bifidobacterium longum and a Bifidobacterium lactis, specifically Bifidobacterium longum ATCC CNCM I-2618, and Bifidobacterium lactis CNCM I-3446, optionally with Bifidobacterium longum ATCC BAA-999, can prevent inflammation-induced intestinal barrier permeability and effect an increase in production or expression of anti-inflammatory cytokines, such as IL-10. The probiotic combinations of the present invention are therefore useful for providing an effective therapy for the treatment or prevention of allergic disorders and/or food intolerance.

The probiotic combinations of the present invention may be employed in compositions for the treatment or prevention of an allergic disorder and/or a food intolerance.

The invention provides:

A method for the treatment or prevention of an allergic disorder and/or food intolerance, comprising administering a probiotic combination as described herein, to a subject e.g. An individual suffering from, or susceptible to, such a disorder or intolerance.

A probiotic combination for use in the treatment or prevention of an allergic disorder and/or food intolerance wherein the probiotic combination comprises Bifidobacterium longum CNCM I-2618 and Bifidobacterium lactis CNCM I-3446.

The probiotic combination may further comprise Bifidobacterium longum ATCC BAA-999.

A probiotic combination for use as described herein, by reinforcing gut barrier and gut barrier integrity and more preferably, by preventing inflammation-induced intestinal barrier permeability to limit allergen sensitization.

A probiotic combination for use as described herein, wherein the allergic disorder or food intolerance is modulated by anti-inflammatory cytokines, preferably wherein the allergic disorder is modulated by IL-10; or wherein the allergic disorder is modulated by T regulatory cells.

A probiotic combination for use as described herein, wherein the allergic disorder or food intolerance is modulated by an imbalance of beneficial bacteria, such as bifidobacteria and lactobacilli; or wherein the allergic disorder or food intolerance is modulated by oxidative stress or inflammatory markers such as CRP.

A probiotic combination as described herein for: decreasing or suppressing the production or expression of Th2 cytokines such as (but not limited to) IL-4 and IL-5 in an allergic disorder and/or food intolerance; for increasing the production or expression of an anti-inflammatory cytokine in an allergic disorder and/or food intolerance, preferably wherein the anti-inflammatory cytokine is IL-10, and more preferably for regulating the concentration ratios of Th1 and Th2 cytokines2 in an allergic disorder and/or food intolerance; for increasing the population of beneficial bacteria, such as bifidobacteria and lactobacilli, in the gut in an allergic disorder and/or food intolerance; for reducing oxidative stress or inflammatory markers such as CRP in an allergic disorder and/or food intolerance; for supporting or promoting tissue healing or for reinforcing gut barrier in an allergic disorder and/or food intolerance; for modulating T regulatory cells and/or B regulatory cells in an allergic disorder and/or food intolerance, preferably for promoting T regulatory cell activation.

A probiotic combination for use as described herein, wherein the allergic disorder is a food allergy, a respiratory allergy, a dermatological allergy.

A probiotic combination for use as described herein, wherein the allergic disorder is: rhinitis, asthma, dermatitis, atopic dermatitis, contact dermatitis, eczema, atopic eczema, urticaria, psoriasis, eosinophilic oesophagitis or a eosinophilic/mast cell-associated gastrointestinal disease.

A probiotic combination for use as described herein wherein the allergen trigger in the allergic disorder or food intolerance is selected from one or more of: a food allergen, dust mite, pollen, molds or mold spores, weed pollen, tree pollen, grass pollen, fleas, pet hair, feathers or pet dander.

A probiotic combination for use as described herein wherein the allergen trigger in the allergic disorder or food intolerance is a food allergen, preferably wherein the food allergen is selected from: a nut, tree nut, peanut, fish, shellfish, mollusks, crustaceans, milk, egg, soy, gluten, cereals, wheat, oats, barley, rye, celery, corn, lupin, sulphites, sesame, mustard, rice, poultry and meat.

A probiotic composition for use as described herein wherein the probiotic composition increases the concentration or expression of an anti-inflammatory cytokine, and more preferably, wherein the anti-inflammatory cytokine is IL-10.

A probiotic combination for use as described herein, wherein the subject is a mammal, preferably a human or a companion animal, preferably wherein the subject is a child, an infant, an adolescent or an adult human, a dog, a puppy, a cat or a kitten.

A probiotic combination for use as described herein, wherein each probiotic in the probiotic combination is administered to a subject in an amount equating to 10⁸ to 10¹² cfu per day.

A probiotic combination for use as described herein, wherein the probiotic combination is administered in the form of a composition. The composition may be selected from the group consisting of: a pharmaceutical formulation, a veterinary formulation, a nutritional formulation, a tube-feed formulation, a dietary supplement, a functional food, a beverage product and a pet care product.

A composition comprising a probiotic combination, as described herein, wherein the probiotic combination comprises Bifidobacterium longum CNCM I-2618 and Bifidobacterium lactis CNCM I-3446, for use in the treatment or prevention of an allergic disorder or for treating or preventing food intolerance. Said probiotic combination may further comprise Bifidobacterium longum ATCC BAA-999. The composition may comprise each probiotic in the probiotic combination in an amount equating to 10⁸ to 10¹² cfu per day. The composition may be selected from the group consisting of: a pharmaceutical formulation, a veterinary formulation, a nutritional formulation, a tube-feed formulation, a dietary supplement, a functional food, a beverage product and a pet care product.

A probiotic combination for use in the manufacture of a composition for use in the treatment or prevention of an allergic disorder and/or food intolerance, wherein said probiotic combination comprises Bifidobacterium longum CNCM I-2618 and Bifidobacterium lactis CNCM I-3446. Said probiotic combination may further comprise Bifidobacterium longum ATCC BAA-999.

A method for treating or preventing an allergic disorder and/or a food intolerance in a subject comprising the step of administering to said subject a probiotic combination, wherein the probiotic combination is Bifidobacterium longum CNCM I-2618 and Bifidobacterium lactis CNCM I-3446. The probiotic combination may further comprise Bifidobacterium longum ATCC BAA-999.

### DESCRIPTION OF THE FIGURES

FIG. 1: Cytokine (IL-10) production in peripheral blood mononuclear cells stimulated with different probiotic strains and combinations

FIG. 2: Prevention of inflammation-induced barrier permeability with different probiotic strains and combinations

### DETAILED DESCRIPTION OF THE INVENTION

The following terms and definitions are used herein:

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

“Infant” refers to a child under the age of 12 months.

“Infant formula” refers to foodstuff intended for the complete nutrition of infants in the context of absence of breast-feeding during the first four to six months of life or a foodstuff for use infants as a complement to other foodstuffs up to the age of 12 months. Typically the term refers to foodstuff intended for the complete nutrition of infants in the context of absence of breast-feeding during the first four to six months of life.

“Probiotic” refers to a microbial cell preparation or components of microbial cells with a beneficial effect on the health or well-being of the host.

A “child” refers to a person above the age of 12 months, but below the age of 10 years.

“Adolescent” refers to a person between the ages of 10-19 (based on the World Health Organisation (WHO) definition).

An “adult” refers to a person aged 20 or more.

A “puppy” refers to a dog that is less than 12 months old.

A “kitten” refers to a cat that is less than 12 months old.

“Cfu” refers to colony forming units, and is measured on a dry weight basis, unless otherwise indicated.

The term Bifidobacterium longum (B. longum) CNCM I-2618 is used interchangeably with Bifidobacterium longum (B. longum) NCC2705.

The term Bifidobacterium lactis (B. lactis) CNCM I-3446 is used interchangeably with Bifidobacterium lactis (B. lactis) NCC2818.

The term Bifidobacterium longum (B. longum) ATCC BAA-999 is used interchangeably with Bifidobacterium longum (B. longum) NCC3001.

The expression “reinforcement of intestinal barrier”, may encompass one or several of the following:
Improved barrier repair, such as (but not limited to) recovery of the integrity of the gastrointestinal barrier, such as repair of a disrupted barrier, reduction of permeability upon inflammatory challenge of the gastrointestinal mucosa, and mucosal repair.
Improved barrier maturation, such as (but not limited to) maturation and/or development of the barrier of an infant, child, adolescent, adult, dog, puppy, cat or kitten.
Improved barrier structure, such as (but not limited to) strengthening of the gastrointestinal barrier, integrity of the gastrointestinal barrier, tight junction structure, and intestinal epithelial lining integrity.
Improved barrier function, such as improvement of gastrointestinal barrier resistance, reduction of gastrointestinal barrier permeability, such as a reduction in penetration of allergens from luminal sites to the mucosa, such as a reduction in transfer of toxic compounds from luminal sites to the mucosa, and reduction of disease susceptibility.
Improved barrier protection, such as (but not limited to) prevention of barrier dysfunction, prevention of barrier leakiness, protection of tight junction structure, protection of the intestinal epithelial lining integrity.

The terms B. longum CNCM I-2618, B. longum CNCM I-2618 and B. longum ATCC BAA-999 are intended to include the bacterium, parts of the bacterium and/or a growth medium fermented by the bacterium.

The B. longum CNCM I-2618, B. longum CNCM I-2618 and B. longum ATCC BAA-999, may each be used as living bacterium as well as inactivated non-replicating bacterial species. “Non-replicating” means that no viable cells and/or colony forming units can be detected by classical plating methods. Such classical plating methods are summarized in the microbiology book: James Monroe Jay, Martin J. Loessner, David A. Golden. 2005. Modern food microbiology. 7th edition, Springer Science, New York, N. Y. 790 p. Typically, the absence of viable cells can be shown as follows: no visible colony on agar plates or no turbidity in liquid growth medium after inoculation with different concentrations of bacterial preparations (“non replicating’ samples”) and incubation under appropriate conditions (aerobic and/or anaerobic atmosphere for at least 24 h).

It is preferred that at least part of the B. longum CNCM I-2618, B. longum CNCM I-2618 and (when present) B. longum ATCC BAA-999, are alive in the combination or composition and preferably arrive alive in the intestine. This way they can persist in the intestine, and be metabolically active. This may increase their effectiveness. They may also be effective by interacting with the commensal bacteria and/or the host. For special sterile food products or medicaments, for example, it might be preferable that B. longum CNCM I-2618, B. longum CNCM I-2618 and (when present) B. longum ATCC BAA-999 are present in a non-replicating form in the combination or composition. Hence, in one embodiment of the present invention at least a part of the B. longum CNCM I-2618, B. longum CNCM I-2618 and (when present) B. longum ATCC BAA-999, are non-replicating in the combination or composition.

In one embodiment, the present invention provides a probiotic combination for use in the treatment or prevention of an allergic disorder and/or food intolerance, wherein the probiotic combination comprises Bifidobacterium longum CNCM I-2618 and Bifidobacterium lactis CNCM I-3446. Optionally, the probiotic combination can further comprise Bifidobacterium longum ATCC BAA-999.

In another embodiment the present invention provides a probiotic combination comprising B. longum CNCM I-2618 and B. lactis CNCM I-3446, and optionally further comprising B. longum ATCC BAA-999, for use in increasing the production or expression of an anti-inflammatory cytokine (preferably IL-10) or regulating the serum concentration ratios of an anti-inflammatory cytokine for example IL-10.

In another embodiment, the present invention provides a probiotic combination comprising B. longum CNCM I-2618 and B. lactis CNCM I-3446, and optionally further comprising B. longum ATCC BAA-999, for use in modulating T regulatory cells.

In another embodiment, the present invention provides a probiotic combination comprising B. longum CNCM I-2618 and B. lactis CNCM I-3446, and optionally further comprising B. longum ATCC BAA-999, for use in the treatment or prevention of an allergic disorder and/or a food intolerance by increasing the population of beneficial bacteria, such as bifidobacteria and lactobacilli, in the gut.

In another embodiment, the present invention provides a probiotic combination comprising B. longum CNCM I-2618 and B. lactis CNCM I-3446, and optionally further comprising B. longum ATCC BAA-999, for use in the treatment or prevention of an allergic disorder and/or food intolerance by reducing oxidative stress or inflammatory markers such as CRP.

In another embodiment, the present invention provides a probiotic combination comprising B. longum CNCM I-2618 and B. lactis CNCM I-3446, and optionally further comprising B. longum ATCC BAA-999, for use in the treatment or prevention of an allergic disorder and/or a food intolerance by supporting or promoting tissue healing or by reinforcing gut barrier integrity and/or function.

The probiotic combination comprising B. longum CNCM I-2618 and B. lactis CNCM I-3446, and optionally further comprising B. longum ATCC BAA-999 is preferably for use in the treatment or prevention of an allergic disorder and/or a food intolerance, preferably the allergic disorder or food intolerance is modulated by anti-inflammatory cytokines e.g. IL-10. The allergic disorder or food intolerance may be one that may be modulated by IL-10 and/or by T regulatory cells differentiation.

In any aspect or embodiment of the present invention, the allergic disorder and/or food intolerance may be modulated by an imbalance of beneficial bacteria, such as bifidobacteria and lactobacilli and/or may be modulated by oxidative stress or inflammatory markers such as CRP.

The present invention further provides a probiotic combination comprising B. longum CNCM I-2618 and B. lactis CNCM I-3446, and optionally further comprising B. longum ATCC BAA-999, for use in the treatment or prevention of an allergic disorder and/or a food intolerance. An allergic disorder may be a food allergy, a respiratory allergy or a dermatological allergy. Examples of specific allergic disorders may be rhinitis, asthma, dermatitis, atopic dermatitis, contact dermatitis, eczema, atopic eczema, urticaria, psoriasis, eosinophilic oesophagitis or an eosinophilic/mast cell-associated gastrointestinal disease.

The probiotic combination of any aspect or embodiment of the present invention may be for use in the treatment or prevention of an allergic disorder, wherein the allergen trigger is an environmental allergen, preferably selected from one or more of: dust mite, pollen, molds or mold spores, weed pollen, tree pollen, grass pollen, fleas, pet hair, feathers or pet dander.

Preferably, the treatment or prevention of an allergic disorder and/or a food intolerance using the probiotic combination of the invention comprises increasing the production or expression of an anti-inflammatory cytokine (preferably IL-10).

The subject to be treated is preferably a mammal, preferably a human or a companion animal (pet), preferably wherein the subject is a child, an infant, an adolescent or an adult human, a dog, a puppy, a cat or a kitten.

In another embodiment, the probiotic combination is for use in the treatment or prevention of a food allergy and/or a food intolerance, wherein the allergen trigger in the allergic disorder or food intolerance is preferably selected from: a nut, tree nut, peanut, fish, shellfish, molluscs, crustaceans, milk, egg, soy, gluten, cereals, wheat, oats, barley, rye, celery, corn, lupin, sulphites, sesame, mustard, rice, poultry and meat. In this embodiment, the treatment or prevention of a food allergy or a food intolerance is preferably in humans.

Alternatively, the probiotic combination for use according to any aspect or embodiment of the present invention is for use in the treatment or prevention of a food allergy and/or a food intolerance in companion animals (pets), preferably a dog or a cat, and more preferably a dog, wherein the allergen trigger in the allergic disorder or food intolerance is selected from meat such as lamb, beef, poultry, pork; fish; crustaceans; corn, rice; wheat; potato; milk and eggs.

Thus, the present invention further provides a probiotic combination comprising B. longum CNCM I-2618 and B. lactis CNCM I-3446, and optionally further comprising B. longum ATCC BAA-999 for treatment of a feline or canine food allergy and/or food intolerance. The probiotic combination is preferably in the form of a composition, more preferably a pet food (particularly a dry pet food) or a pet nutritional supplement or a veterinary composition (particularly a tablet, a capsule or a dry powder).

In another embodiment, the subject to be treated is a dog, puppy, cat or kitten. Thus, the present invention further provides a probiotic combination comprising B. longum CNCM I-2618 and B. lactis CNCM I-3446, and optionally further comprising B. longum ATCC BAA-999 for treatment or prevention of an allergic disorder e.g. a food allergy and/or a food intolerance in a companion animal, preferably a dog or a cat. The probiotic combination is preferably in the form of a composition, more preferably a pet food, a pet nutritional supplement or a veterinary composition.

In yet another embodiment, the subject to be treated in a child, an infant, an adolescent or an adult human. Thus, the present invention further provides a probiotic combination comprising B. longum CNCM I-2618 and B. lactis CNCM I-3446, and optionally further comprising B. longum ATCC BAA-999 for the treatment or prevention of a food allergy or a food intolerance in these subjects. The probiotic combination is preferably in the form of a composition, more preferably a food, a nutritional supplement or a pharmaceutical composition (particularly a tablet, a capsule, granules, or a dry powder).

In any embodiment of the present invention, the probiotic combination may contain B. longum CNCM I-2618 and B. lactis CNCM I-3446 as the only probiotic bacteria. The probiotic combination is preferably in the form of a composition, more preferably a food, a nutritional supplement or a pharmaceutical or veterinary composition.

In any embodiment of the present invention, the probiotic combination may comprise B. longum CNCM I-2618, B. lactis CNCM I-3446 and B. longum ATCC BAA-999. Alternatively, the probiotic combination according to any embodiment of the present invention may comprise B. longum CNCM I-2618, B. lactis CNCM I-3446 and B. longum ATCC BAA-999 as the only probiotic bacteria. The probiotic combination is preferably in the form of a composition, more preferably a food, a nutritional supplement or a pharmaceutical or veterinary composition.

The probiotic combination may be in the form of a composition as described in any embodiment, wherein the composition contains B. longum CNCM I-2618 and B. lactis CNCM I-3446 as the only probiotic bacteria. The probiotic composition is preferably in the form of a food, a nutritional supplement or a pharmaceutical or veterinary composition.

The probiotic combination may be in the form of a composition as described in any embodiment, wherein the composition comprises B. longum CNCM I-2618, B. lactis CNCM I-3446 and B. longum ATCC BAA-999. Alternatively, the probiotic combination may be in the form of a composition as described in any embodiment, wherein the composition contains B. longum CNCM I-2618, B. lactis CNCM I-3446 and B. longum ATCC BAA-999 as the only probiotic bacteria. The probiotic composition is preferably in the form of a food, a nutritional supplement or a pharmaceutical or veterinary composition.

Any suitable dose of the probiotic combination may be used. Preferably, in any embodiment of the invention, the probiotic combination comprises B. longum CNCM I-2618 and B. lactis CNCM I-3446, and optionally further comprising B. longum ATCC BAA-999, wherein each probiotic is administered to a subject in an amount equating to 10⁷ to 10¹² cfu per day.

Although the probiotic components of the combination can be used without further processing, the probiotic combination according to any embodiment of the invention is preferably administered in the form of a composition. Suitable compositions comprise B. longum CNCM I-2618 and B. lactis CNCM I-3446, and optionally further comprising B. longum ATCC BAA-999 and may preferably be in the form of a pharmaceutical or veterinary formulation comprising one or more pharmaceutically or veterinary acceptable excipients, a nutritional formulation (e.g. including a nutritional supplement), a tube-feed formulation, a dietary supplement, a functional food, a beverage product and a pet care product (e.g. a pet food, or a pet nutritional supplement).

The pharmaceutical or veterinary formulation may be in the form of a tablet, a capsule, granules, or a powder.

According to any embodiment of the present invention, the composition may comprise an amount of each probiotics equating to 10⁷ to 10¹² cfu per day. This amount may either be as a single dose, or spread across multiple doses.

Also provided is a probiotic combination comprising B. longum CNCM I-2618 and B. lactis CNCM I-3446, and optionally further comprising B. longum ATCC BAA-999, for use in the manufacture of a composition for use in the treatment or prevention of an allergic disorder e.g. a food allergy, and/or a food intolerance.

The invention further provides a method for treatment or prevention of an allergic disorder e.g. a food allergy and/or a food intolerance in a subject comprising the step of administering to said subject a probiotic combination, wherein the probiotic combination comprises B. longum CNCM I-2618 and B. lactis CNCM I-3446, and optionally further comprises B. longum ATCC BAA-999.

The probiotic combination of the present invention comprising B. longum CNCM I-2618 and B. lactis CNCM I-3446, and optionally further comprising B. longum ATCC BAA-999, may be provided for simultaneous or sequential administration of each of the probiotics. Alternatively, the probiotic combination may be formulation as a single composition.

The probiotic combination comprising Bifidobacterium longum CNCM I-2618 and Bifidobacterium lactis CNCM I-2446 and optionally further comprising B. longum ATCC BAA-999, may be administered as a composition (e.g. a capsule, a tablet, granules or a powder) containing, for example, 10⁷-10¹² colony forming units (cfu) of each probiotic component, or may be incorporated in a nutritional composition such as a nutritionally complete formula (for example an infant formula or a clinical nutrition product), a dairy product, a beverage powder, a dehydrated soup, a dietary supplement, a meal replacement, a nutritional bar, a cereal, a confectionery product or a dry pet food.

In one embodiment, the combination may be in the form of a single capsule comprising both B. longum CNCM I-2618 and B. lactis CNCM I-2446, or a single capsule comprising B. longum CNCM I-2618, B. lactis CNCM I-2446 and B. longum ATCC BAA-999.

Alternatively, the combination may be provided as separate capsules, comprising B. longum CNCM I-2618 in one capsule and B. lactis CNCM I-2446 in another capsule, for simultaneous or sequential administration; or the combination may be provided as separate capsules comprising B. longum CNCM I-2618, B. lactis CNCM I-2446 and B. longum ATCC BAA-999 as separate capsules for simultaneous or sequential administration.

When incorporated in a nutritional composition, B. longum CNCM I-2618 and B. lactis CNCM I-3446 and optionally B. longum ATCC BAA-999 may each be present in the composition in an amount equivalent to between 5×10⁴ and 10¹⁹ cfu/g (dry weight). These expressions of quantity include the possibilities that the bacteria are live, inactivated or dead or even present as fragments such as DNA or cell wall materials or as metabolites. In other words, the quantities of bacteria are expressed in terms of the colony forming ability of that quantity of bacteria as if all the bacteria were live irrespective of whether they are, in fact, live, inactivated or dead, fragmented or a mixture of any or all of these states. Preferably each of the B. longum CNCM I-2618, B. lactis CNCM I-3446 and B. longum ATCC BAA-999 (when present) is present in an amount equivalent to between 5×10⁴ to 10⁹, more preferably 10⁷ to 10⁹ cfu/g of dry composition.

In embodiments of the present invention wherein the probiotic combination further comprises B. longum ATCC BAA-999, the B. longum ATCC BAA-999 may be present either in the same composition or in a separate composition for simultaneous or sequential administration. For example, in the above described capsules, the B. longum ATCC BAA-999 may be enclosed in capsules with the B. longum CNCM I-2618 and B. lactis CNCM I-3446, wherein each capsule contains 10⁷ to 10⁹ of each strain colony forming units (cfu). Likewise, the composition comprising B. longum CNCM I-2618, B. lactis CNCM I-3446 and B. longum ATCC BAA-999 may be incorporated in a nutritional composition such as a nutritionally complete formula (for example an infant formula or a clinical nutrition product), a dairy product, a beverage powder, a dehydrated soup, a dietary supplement, a meal replacement, a nutritional bar, a cereal, a confectionery product or a dry pet food.

B. longum CNCM I-2618 and B. lactis CNCM I-3446 may each be cultured according to any suitable method and prepared for encapsulation or addition to a nutritional composition by freeze-drying or spray-drying for example. Alternatively, they may be purchased already prepared in a suitable form for addition to food products.

ATCC BAA-999 is commercially available and may be obtained from Morinaga Milk Industry Co. Ltd. of Japan under the trade mark BB536. It may be cultured according to any suitable method and prepared for encapsulation or addition to a nutritional composition by freeze-drying or spray-drying for example. Alternatively, it may be purchased already prepared in a suitable form for addition to food products.

A nutritionally complete formula for use in the present invention may comprise a source of protein, preferably a dietary protein such as an animal protein (for example milk, meat or egg protein), a vegetable protein (for example soy, wheat, rice or pea protein); mixtures of free amino acids; or combinations thereof. Milk proteins such as casein and whey protein and soy proteins are particularly preferred. The composition may also contain a source of carbohydrates and a source of fat.

If the formula includes a fat source, it preferably provides 5% to 55% of the energy of the formula; for example 20% to 50% of the energy. The lipids making up the fat source may be any suitable fat or fat mixture. Vegetable fats such as soy oil, palm oil, coconut oil, safflower oil, sunflower oil, corn oil, canola oil, and lecithins are particularly suitable. Animal fats such as milk fat may also be added if desired.

If the formula includes a carbohydrate source, it preferably provides 40% to 80% of the energy of the formula. Any suitable carbohydrate may be used, for example sucrose, lactose, glucose, fructose, corn syrup solids, maltodextrins, and mixtures thereof. Dietary fibre may also be added if desired. The dietary fibre may be from any suitable origin, including for example soy, pea, oat, pectin, guar gum, gum Arabic, fructo-oligosaccharides, galacto-oligosaccharides, sialyl-lactose and oligosaccharides derived from animal milks. Suitable vitamins and minerals may be included in the nutritional formula in an amount to meet the appropriate guidelines.

The compositions of the present invention may further include a prebiotic. Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such as fructo-oligosaccharides (FOS), inulin, xylo-oligosaccharides (XOS), polydextrose or any mixture thereof. In a particular embodiment, the prebiotics may be fructo-oligosaccharides and/or inulin. An example is a combination of 70% short chain fructo-oligosaccharides and 30% inulin, which is registered by Nestle under the trademark “Prebio 1”.

One or more food grade emulsifiers may be incorporated into the nutritional formula if desired; for example diacetyl tartaric acid esters of mono- and di-glycerides, lecithin and mono- and di-glycerides. Similarly suitable salts and stabilisers may be included.

The nutritionally complete formula may be prepared in any suitable manner. For example, the protein source, the carbohydrate source, and the fat source may be blended together in appropriate proportions. If used, the emulsifiers may be included in the blend. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 3 seconds to about 5 minutes. This may be carried out by steam injection or by heat exchanger; for example a plate heat exchanger or a tubular heat exchanger.

The liquid mixture may then be cooled to a temperature in the range from about 60° C. to about 85° C.; for example by flash cooling. The liquid mixture may then be homogenised; for example in two stages at about 10 MPa to about 30 MPa in the first stage and about 2 MPa to about 10 MPa in the second stage. The homogenised mixture may then be further cooled to add any heat sensitive components; such as vitamins and minerals. The pH and solids content of the homogenised mixture is conveniently standardised at this point.

The homogenised mixture may then be transferred to a suitable drying apparatus such as a spray dryer or freeze dryer and converted to powder. The powder should have a moisture content of less than about 5% by weight. The B. longum CNCM I-2618, and/or B. lactis CNCM I-3446, and/or B. longum ATCC BAA-999 may be added to the powder in the desired quantity by dry mixing.

A dry pet food for use in the present invention may include any one or more of a carbohydrate source, a protein source and lipid source.

Any suitable carbohydrate source may be used. Preferably the carbohydrate source is provided in the form of grains, flours or starches. For example, the carbohydrate source may be rice, barley, sorghum, millet, oat, corn meal or wheat flour. Simple sugars such as sucrose, glucose and corn syrups may also be used. The amount of carbohydrate provided by the carbohydrate source may be selected as desired. For example, the pet food may contain up to about 60% by weight of carbohydrate.

Suitable protein sources may be selected from any suitable animal or vegetable protein source; for example muscular or skeletal meat, meat and bone meal, poultry meal, fish meal, milk proteins, corn gluten, wheat gluten, soy flour, soy protein concentrates, soy protein isolates, egg proteins, whey, casein, gluten, and the like. For elderly animals, it is preferred for the protein source to contain a high quality animal protein. The amount of protein provided by the protein source may be selected as desired. For example, the pet food may contain about 12% to about 70% by weight of protein on a dry basis.

The pet food may contain a fat source. Any suitable fat source may be used. Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used. In addition to essential fatty acids (linoleic and alpha-linoleic acid) the fat source may include long chain fatty acids. Suitable long chain fatty acids include gamma linoleic acid, stearidonic acid, arachidonic acid, eicosapentanoic acid, and docosahexanoic acid. Fish oils are a suitable source of eicosapentanoic acids and docosahexanoic acid. Borage oil, blackcurrant seed oil and evening primrose oil are suitable sources of gamma linoleic acid. Rapeseed oil, soybean oil, linseed oil and walnut oil are suitable sources of alpha-linolenic acid. Safflower oils, sunflower oils, corn oils and soybean oils are suitable sources of linoleic acid. Olive oil, rapeseed oil (canola), high oleic sunflower oil, safflower oil, peanut oil, and rice bran oil are suitable sources of monounsaturated fatty acids. The amount of fat provided by the fat source may be selected as desired. For example, the pet food may contain about 5% to about 40% by weight of fat on a dry basis. Preferably, the pet food has a relatively reduced amount of fat.

The choice of the carbohydrate, protein and lipid sources is not critical and will be selected based upon nutritional needs of the animal, palatability considerations, and the type of product produced. Further, various other ingredients, for example, sugar, salt, spices, seasonings, vitamins, minerals, flavouring agents, gums, and probiotic microorganisms may also be incorporated into the pet food as desired.

For elderly pets, the pet food preferably contains proportionally less fat than pet foods for younger pets. Further, the starch sources may include one or more of oat, rice, barley, wheat and corn.

The pet food may be produced by extrusion cooking, although baking and other suitable processes may be used. When extrusion cooked, the pet food is usually provided in the form of a kibble. The probiotic components may preferably be coated onto or filled into the dried pet food. A suitable process is described in European Patent Application No 0862863.

The probiotic combination of present invention, and compositions thereof may be used to treat, manage or prevent an allergic disorder and/or a food intolerance in individuals who having an allergic disorder or food intolerance, or individuals that are susceptible to an allergic disorder or food intolerance (e.g. individuals having an atopic history).

Typically, the composition may be selected from the group consisting of a food composition, a pet food composition, a dietary supplement, a nutraceutical, a nutritional formula, a drink, and/or a medical composition.

Examples of food compositions that are applicable to the present invention are yoghurts, milk, flavoured milk, ice cream, ready to eat desserts, powders for re-constitution with, e.g., milk or water, chocolate milk drinks, malt drinks, ready-to-eat dishes, instant dishes or drinks for humans or food compositions representing a complete or a partial diet intended for pets or livestock. Consequently, in one embodiment the composition according to the present invention is a food product intended for humans, pets or livestock, and preferably humans and pets. In a preferred embodiment, the composition is a food product or a dietary supplement intended for humans (infant, child, adolescent, or adult) or companion animals (pets) (preferably dog, puppy, cat or kitten).

The composition of the present invention may further contain protective hydrocolloids (such as gums, proteins, modified starches), binders, film forming agents, encapsulating agent(s)/material(s), wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, lignin sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

The composition may be a nutritionally complete formula. The composition according to the invention may comprise a source of protein.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof.

The proteins may be intact or hydrolysed or a mixture of intact and hydrolysed proteins. It may be desirable to supply partially hydrolysed proteins (degree of hydrolysis between 2 and 20%), for example for human subjects and/or animals at risk of developing cows' milk allergy.

Furthermore, pre-hydrolysed protein sources are generally easier digested and absorbed by an impaired gastro-intestinal tract.

If hydrolysed proteins are required, the hydrolysis process may be carried out as desired and as is known in the art. It may be desirable to supply partially hydrolysed proteins (degree of hydrolysis between 2 and 20%).

For example, a whey protein hydrolysate may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

The composition may also contain a source of carbohydrates and a source of fat. If the composition includes a fat source, the fat source preferably provides 5% to 40% of the energy of the composition; for example 20% to 30% of the energy. A suitable fat profile may be obtained using a blend of canola oil, corn oil and high-oleic acid sunflower oil.

A source of carbohydrate may be added to the composition.

The source of carbohydrates preferably provides 40% to 80% of the energy of the composition. Any suitable carbohydrate may be used, for example sucrose, lactose, glucose, fructose, corn syrup solids, maltodextrins, and mixtures thereof. Dietary fibre may also be added if desired. Dietary fibre passes through the small intestine undigested by enzymes and functions as a natural bulking agent and laxative. Dietary fibre may be soluble or insoluble and in general a blend of the two types is preferred. Suitable sources of dietary fibre include soy, pea, oat, pectin, guar gum, partially hydrolysed guar gum, gum Arabic, fructo-oligosaccharides, acidic oligosaccharides, galacto-oligosaccharides, sialyl-lactose and oligosaccharides derived from animal milks. A preferred fibre blend is a mixture of inulin with shorter chain fructo-oligosaccharides. Preferably, if fibre is present, the fibre content is between 2 and 40 g/l of the composition as consumed, more preferably between 4 and 10 g/I.

The composition may also contain minerals and micronutrients such as trace elements and vitamins in accordance with the recommendations of Government bodies such as the USRDA. For example, the composition may contain per daily dose one or more of the following micronutrients in the ranges given: 300 to 500 mg calcium, 50 to 100 mg magnesium, 150 to 250 mg phosphorus, 5 to 20 mg iron, 1 to 7 mg zinc, 0.1 to 0.3 mg copper, 50 to 200 μg iodine, 5 to 15 μg selenium, 1000 to 3000 μg beta carotene, 10 to 80 mg Vitamin C, 1 to 2 mg Vitamin B1, 0.5 to 1.5 mg Vitamin B6, 0.5 to 2 mg Vitamin B2, 5 to 18 mg niacin, 0.5 to 2.0 μg Vitamin B12, 100 to 800 μg folic acid, 30 to 70 μg biotin, 1 to 5 μg Vitamin D, 3 to 10 μg Vitamin E.

One or more food grade emulsifiers may be incorporated into the composition if desired; for example diacetyl tartaric acid esters of mono- and di-glycerides, lecithin and mono- and di-glycerides. Similarly suitable salts and stabilisers may be included.

The composition may be orally and/or enterally administrable; for example in the form of a powder for re-constitution with milk or water.

The compositions are administered in an amount sufficient to at least partially treat or arrest the symptoms of the allergic disorder or food intolerance and its complications. An amount adequate to accomplish this is defined as “a therapeutically effective dose”. Amounts effective for this purpose will depend on a number of factors known to those of skill in the art such as the severity of the disease and the weight and general state of the patient.

In prophylactic applications, compositions according to the invention are administered to a patient susceptible to or otherwise at risk of a particular disease in an amount that is sufficient to at least partially reduce the risk of developing a disease. Such an amount is defined to be “a prophylactic effective dose”. Again, the precise amounts depend on a number of patient specific factors such as the patient's state of health and weight.

Generally, B. longum CNCM I-2618, B. lactis CNCM I-3446 and (where present) B. longum ATCC BAA-999, will each be administered in a therapeutically effective dose and/or in a prophylactic effective dose.

If B. longum CNCM I-2618, B. lactis CNCM I-3446 and (where present) B. longum ATCC BAA-999 are present in a viable form, it is theoretically effective in any concentration considering the fact that these bacteria can colonize the gut and multiply. For the compositions of the present invention, it is generally preferred that a daily dose of the composition comprises between 10⁴ and 10¹² cfu of each of the probiotic agents. A particular suitable daily dose of each of the probiotics is from 10³ to 10¹² cfu.

In the case of inactivated and/or non-replicating B. longum CNCM I-2618, B. lactis CNCM I-3446, and (where present) B. longum ATCC BAA-999, it is generally preferred that the composition of the present invention comprises between 10² and 10¹² non-replicating cells of Bifidobacterium longum ATCC BAA-999, per gram of the dry weight of the composition. A particular suitable dose of each of the probiotics, is from 10³ to 10¹² non-replicating cells, more preferably from 10⁵ to 10⁸ non-replicating cells per gram of the dry weight of the composition.

Obviously, non-replicating micro-organisms do not form colonies, consequently, the term cells is to be understood as the amount of non replicating micro-organisms that is obtained from the specified amount of replicating bacterial cells. This includes micro-organisms that are inactivated, non-viable or dead or present as fragments such as DNA or cell wall materials.

The composition of the present invention may be provided in powder form having a water activity of lower than 0.2, for example in the range of 0.19-0.05, preferably smaller than 0.15.

The composition may be a shelf stable powder. The low water activity provides this shelf stability and ensures that probiotic micro-organisms, will remain viable even after long storage times.

Water activity or a_{w} is a measurement of the energy status of the water in a system. It is defined as the vapour pressure of water divided by that of pure water at the same temperature; therefore, pure distilled water has a water activity of exactly one.

Additionally or alternatively, the probiotic micro-organism B. longum CNCM I-2618, B. lactis CNCM I-3446 and (where present) B. longum ATCC BAA-999 may be provided in an encapsulated form.

It has been found that encapsulation of the bacteria has therapeutic and technical advantages. Encapsulation increases the survival of the bacteria and thus the number of live bacteria which arrive in the intestine. Furthermore, the bacteria are gradually released allowing a prolonged action of the bacteria on the health of the subject. Bacteria may be micro-encapsulated, for example as described by FR2443247 (Societe des Produits Nestle), incorporated herein by reference. Briefly, the bacteria may be freeze or spray dried and incorporated into a gel.

The invention will now be further described by the reference to the following example.

### Example

### Preparation of Bacteria:

The day before the assay, three selected bacteria strains from the Nestle Culture Collection NCC 3001, 2818 and 2705 was cultured in 10 ml MRS+cysteine and grown for 16 hrs at 37° C. in anaerobic condition.

Bacterial cultures were centrifuged at 5000 rpm 5 min (room temperature). Bacterial pellet were suspended in cold phosphate buffered saline (PBS) (10 mL). The optical density of each bacterial culture was measured at 600 nm. Adjusted bacteria preparations in RPMI culture medium were set to have 5×10⁶ CFU/ml and 1×10⁷ CFU/ml according to pre-test of bacterial colony forming unit counting on selective agar medium that validated correspondence of OD and CFU.

### Preparation of Peripheral Blood Mononuclear Cell (PBMC):

PBMC isolated from three healthy donors were washed once in PBS. After a centrifugation at 500 g for 5 minutes, the cell pellet was suspended in 2 ml RPMI+10% fetal calf serum (FCS). Cells were counted and preparations adapted to have 2×10⁶ cells/ml.

Stimulation of PBMC with bacteria:

PBMC were seeded in 12 wells culture plate (500 μL) then bacteria preparations were added (500 μL). Co-cultures were incubated for 24 hrs at 37° C. with 10% CO₂.

For the cytokine analyses, the supernatants were centrifuged for 5 minutes at 500 g and transferred in a new tube. The samples were stored at −20° C. until assessment. Cytokine IL-10 was measured by ELISA (IL-10 ELISA, R&D Systems, MN).

### Assessment of Intestinal Barrier Permeability

Caco-2 and HT-29-MTX cells were co-cultured in 12 well cultures plates on polystyrene filter inserts at a ratio of 3:1. Upon differentiation (14 days), co-cultures were pre-incubated with bacteria preparations (5×10⁶) for 24 h prior to basolateral stimulus with TNFα (0.6 ng/mL) and IFNγ (2.5 ng/mL) to alter barrier integrity (control). Trans-epithelial electrical resistance (TEER) was measured after 16 h to quantify inflammation-induced changes in barrier permeability illustrated as percent increase in TEER over TNFα/IFNγ treated controls.

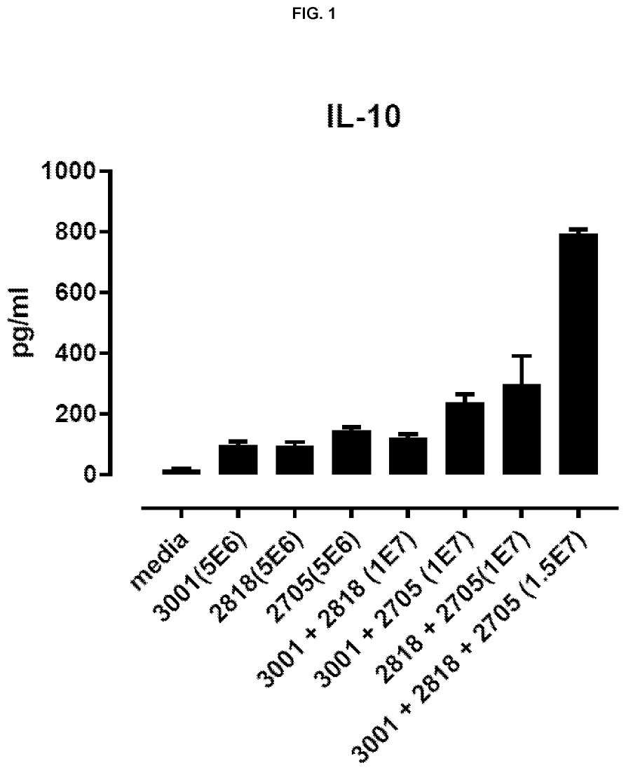

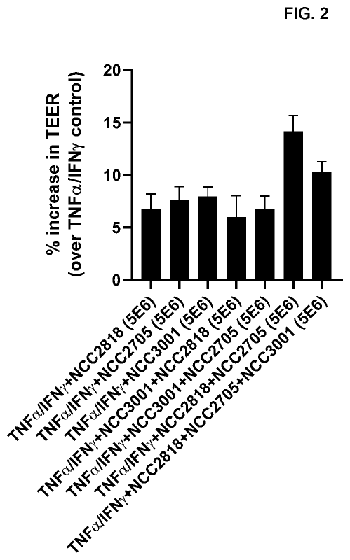

The results are shown in FIGS. 1 and 2. As clearly shown in FIG. 1, a combination of B. longum CNCM I-2618 and B. lactis according to the present invention are surprisingly effective at increasing the production of the anti-inflammatory cytokine IL-10 (FIG. 1).

FIG. 1 further demonstrates a surprising synergistic effect on the increase in the production of the anti-inflammatory cytokine IL-10 when a triple combination of B. longum CNCM I-2618, B. lactis CNCM I-3446 and B. longum ATCC BAA-999 of the invention is employed.

As clearly shown in FIG. 2 and according to the present invention, B. longum CNCM I-2618 and B. lactis CNCM I-3446 are surprisingly effective at preventing inflammation-induced barrier permeability compared to single strains or combinations of two strains. The triple combination of B. longum CNCM I-2618, B. lactis CNCM I-3446 and B. Longum ATCC BAA-999 according to one aspect of the present invention also shows prevention of inflammation-induced barrier disruption (FIG. 2), although to a lesser extent.

1. A method for treating or preventing an allergic disorder or a food intolerance in a subject comprising administering a probiotic combination to the subject, wherein the probiotic combination comprises Bifidobacterium longum CNCM I-2618 and Bifidobacterium lactis CNCM I-3446.

2. The method of claim 1, wherein the probiotic combination further comprises Bifidobacterium longum ATCC BAA-999.

3. The method of claim 1, wherein the allergic disorder or food intolerance is modulated by anti-inflammatory cytokines, wherein the allergic disorder or food intolerance is modulated by IL-10; or wherein the allergic disorder or food intolerance is modulated by T regulatory cells.

4. The method of claim 1, wherein the allergic disorder or food intolerance is modulated by an imbalance of beneficial bacteria, including bifidobacteria or lactobacilli, or wherein the allergic disorder is modulated by oxidative stress or inflammatory markers including CRP.

5. The method of claim 1, wherein the allergic disorder is a food allergy, a respiratory allergy, or a dermatological allergy.

6. The method of claim 1, wherein the allergic disorder is: rhinitis, asthma, dermatitis, atopic dermatitis, contact dermatitis, eczema, atopic eczema, urticaria, psoriasis, eosinophilic oesophagitis or an eosinophilic-associated gastrointestinal disease.

7. The method of claim 1, wherein the probiotic composition increases the concentration or expression of an anti-inflammatory cytokine, and wherein the anti-inflammatory cytokine is IL-10.

8. The method of claim 1, wherein the method includes reinforcing gut barrier and gut barrier integrity of the subject consuming the combination.

9. The method of claim 8, wherein reinforcement of the gut barrier is selected in the group consisting of: prevention of inflammation-induced intestinal barrier dysfunction, reduction of intestinal permeability, improvement of barrier function, reinforcement/protection of intestinal barrier and combinations thereof.

10. The method of claim 1, wherein the subject is a human or a companion animal.

11. The method of claim 1, wherein each probiotic in the probiotic combination is administered to the subject in an amount equating to 10⁸ to 10¹² cfu per day.

12. The method of claim 1, wherein the probiotic combination is administered in the form of a composition.

13. The method of claim 12, wherein the composition is selected from the group consisting of: a pharmaceutical formulation, a veterinary formulation, a nutritional formulation, a tube-feed formulation, a dietary supplement, a functional food, a beverage product and a pet care product.

14-17. (canceled)

18. The method of claim 1, wherein the subject is a child, an infant, an adolescent or an adult human, a dog, a puppy, a cat or a kitten.
